(19) United States
(12) Patent Application Publication
(10) Pub. No.: US 2002/0155109 A1
(43) Pub. Date: Oct. 24, 2002

(54) Bispecific antibodies that bind TRAIL-R1 and TRAIL-R2

(76) Inventor: David H. Lynch, Bainbridge Island, WA (US)

Correspondence Address: IMMUNEX CORPORATION, LAW DEPARTMENT, 51 UNIVERSITY STREET, SEATTLE, WA, 98101

(21) Appl. No.: 09/839,632

(22) Filed: Apr. 20, 2001

(51) Int. Cl. A61K 39/395; C07K 16/30; C07K 16/28

(52) U.S. Cl. 424/143.1; 530/388.22

(57) ABSTRACT

Bispecific antibodies that bind TRAIL receptor 1 and TRAIL receptor 2 are provided. Bispecific antibodies that induce apoptosis of tumor cells and virally infected cells are employed in treating cancer and viral infections.

### BACKGROUND OF THE INVENTION

TNF-related apoptosis-inducing ligand (TRAIL) is a member of the tumor necrosis factor (TNF) family of ligands. TRAIL induces apoptosis of certain transformed cells, including a number of different types of cancer cells as well as virally infected cells, while not inducing apoptosis of a number of normal cell types (Wiley et al., Immunity, 3:673-682, 1995; Walczak et al., Nature Medicine 5:157-163, 1999; and U.S. Pat. No. 5,763,223).

There are four known cell surface receptors for TRAIL, designated TRAIL Receptor 1 (TRAIL-R1, DR4); TRAIL Receptor 2 (TRAIL-R2, DR5, Apo-2, TRICK2, KILLER, TR6, Tango-63); TRAIL Receptor 3 (TRAIL-R3, DcR1, TR5, TRID, LIT) and TRAIL Receptor 4 (TRAIL-R4, DcR2, TRUNDD). TRAIL-R1 is described in WO 98/32856; TRAIL-R2 in U.S. Pat. No. 6,072,047; TRAIL-R3 in WO 99/00423; and TRAIL-R4 in WO 99/03992. In addition, osteoprotegrin (OPG), a soluble (secreted) member of the TNF receptor family of proteins, also binds TRAIL (Emery et al., J. Biol. Chem. 273:14363; 1998). The existence of at least five TRAIL-binding proteins highlights the biological complexity of the TRAIL/TRAIL receptor system.

TRAIL-R1 and TRAIL-R2 are type I transmembrane proteins, containing (from N-terminus to C-terminus) a signal peptide, an extracellular domain, a transmembrane region, and a cytoplasmic (intracellular) domain. The cytoplasmic domains of TRAIL-R1 and TRAIL-R2 each include a so-called death domain. In contrast, TRAIL-R3 lacks a cytoplasmic domain, and is believed to be attached to the cell surface by glycosylphosphatidylinositol (GPI) linkage. TRAIL-R4 has a truncated cytoplasmic domain, which includes only a partial death domain.

TRAIL-R1 and TRAIL-R2 have been reported to transduce an apoptotic signal to TRAIL-sensitive cancer cells, upon binding of TRAIL. In contrast, binding of TRAIL to TRAIL-R3 or TRAIL-R4 is not believed to result in transduction of an apoptotic signal. (See Griffith et al., J. Immunol. 162:2597, 1999; and Degli-Esposti et al., Immunity, 7:813-820, 1997).

### SUMMARY OF THE INVENTION

The present invention provides bispecific antibodies that bind TRAIL Receptor-1 (TRAIL-R1) and TRAIL Receptor-2 (TRAIL-R2). In particular embodiments, the bispecific antibody is capable of inducing apoptosis of cancer cells and virally infected cells. The present invention provides a method for treating cancer, by administering to a cancer patient a bispecific antibody that binds TRAIL-R1 and TRAIL-R2 and induces apoptosis of the cancer cells. A method for treating an individual afflicted with a viral infection comprises administering to the individual a bispecific antibody that binds TRAIL-R1 and TRAIL-R2 and induces apoptosis of virally-infected cells.

### DETAILED DESCRIPTION OF THE INVENTION

The present invention provides bispecific antibodies that bind TRAIL-R1 and TRAIL-R2. Bispecific antibodies (BsAbs) are antibodies that have two different antigen binding sites, such that the antibody specifically binds to two different antigens. Antibodies having higher valencies (i.e., the ability to bind to more than two antigens) can also be prepared; they are referred to as multispecific antibodies.

The bispecific antibody preferably is a monoclonal antibody (MAb). In particular embodiments, the antibody is chimeric, or humanized, or fully human. Fully human antibodies may be generated by procedures that involve immunizing transgenic mice, wherein human immunoglobulin genes have been introduced into the mice, as discussed below. Bispecific antibodies of the invention, which bind TRAIL-R1 and TRAIL-R2, are referred to herein as bispecific R1/R2 antibodies or bispecific R1/R2 MAbs.

TRAIL-R1 is described in WO 98/32856, which is incorporated by reference herein. WO 98/32856 includes DNA and amino acid sequence information for human TRAIL-R1, and describes methods for preparing TRAIL-R1 polypeptides. DNA and amino acid sequence information for human TRAIL-R2, and methods for preparing TRAIL-R2 polypeptides, are disclosed in U.S. Pat. No. 6,072,047, hereby incorporated by reference. TRAIL-R1 and TRAIL-R2 are transmembrane proteins containing an N-terminal extracellular domain, a transmembrane region, and a C-terminal cytoplasmic (intracellular) domain. TRAIL, a member of the tumor necrosis factor (TNF) family of ligands, binds to TRAIL-R1 and TRAIL-R2 (Wiley et al., Immunity, 3:673-682, 1995; U.S. Pat. No. 5,763,223).

The “bispecific antibodies” of the invention encompass antigen-binding fragments of the bispecific R1/R2 antibodies (including monoclonal antibodies) provided herein. One example of such a fragment, which retains the ability to bind TRAIL-R1 and TRAIL-R2, is a F(ab′)₂ fragment. Antigen-binding antibody fragments and derivatives that are produced by genetic engineering techniques are also provided.

Bispecific antibodies that bind TRAIL-R1 and TRAIL-R2 may be screened to identify those that additionally exhibit agonistic (ligand-mimicking) properties. Such antibodies, upon binding to cell surface TRAIL-R1 or TRAIL-R2, induce a biological effect similar to a biological effect induced when TRAIL binds to cell surface TRAIL-R1 or TRAIL-R2. In particular embodiments, agonistic bispecific R1/R2 antibodies induce apoptosis of target cells such as cancer cells or virally infected cells, as has been reported for TRAIL. The ability of TRAIL to kill transformed cells, including cancer cells and virally infected cells, is disclosed in Wiley et al. (Immunity 3:673-682, 1995); and in U.S. Pat. No. 5,763,223.

Bispecific antibodies that bind TRAIL-R1 and TRAIL-R2 may be screened for the ability to kill target cells of interest, using any of a number of conventional assay techniques. Examples of suitable assay procedures are described in the examples section and elsewhere below.

The present invention provides a method for treating a tumor-bearing subject, comprising administering to the subject a bispecific R1/R2 antibody that is capable of killing cancer cells. Also provided herein is a method for treating a subject with a viral infection, comprising administering to the subject a bispecific R1/R2 antibody that is capable of killing virally infected cancer cells.

TRAIL-R1 and TRAIL-R2 have been reported to transduce an apoptotic signal to TRAIL-sensitive cancer cells, upon binding of TRAIL to those receptors. In contrast, binding of TRAIL to TRAIL-R3 or TRAIL-R4 is not believed to result in transduction of an apoptotic signal. (See Griffith et al., J. Immunol. 162:2597, 1999; and Degli-Esposti et al., Immunity, 7:813-820, 1997). Osteoprotegerin (OPG) also binds TRAIL (Emery et al., J. Biol. Chem. 273:14363; 1998). Being a soluble protein (secreted from cells), rather than a cell surface receptor, OPG does not transduce an apoptotic signal to a cell upon TRAIL binding.

Bispecific antibodies that bind to TRAIL-R1 and TRAIL-R2, but lack the ability to bind to at least one of TRAIL-R3, TRAIL-R4, and OPG, are embodiments of the antibodies provided herein. Agonistic R1/R2 antibodies that do not bind to any of TRAIL-R3, TRAIL-R4, and OPG are advantageous for inducing apoptosis of target cells in vivo, since none of the administered dosage will bind to non-signaling receptors (receptors that do not transduce an apoptotic signal).

Various cancer cell lines express different subsets of TRAIL receptors. Some cancer cell types have been reported to express only one of the two apoptosis-mediating receptors, at detectable levels. To illustrate, one study of TRAIL receptor expression on cancer cells is reported in Griffith et al. (J. Immunol. 162:2597, 1999). Griffith et al. studied TRAIL receptor expression on several human melanoma cell lines. Expression patterns varied from cell line to cell line, and cell lines were found to express from one to all four receptors. Regarding the two receptors that mediate transduction of an apoptotic signal, some of the melanoma cell lines expressed only TRAIL-R2, whereas others expressed both TRAIL-R1 and TRAIL-R2.

Griffith and Lynch (Current Opinion in Immunology, 10:559-563, 1998) report an analysis of TRAIL receptor mRNA expression on cancer cells. The study was conducted on a variety of human tumor cell lines, including melanoma, colon carcinoma, breast adenocarcinoma, and lung adenocarcinoma. Griffith and Lynch indicate whether mRNA for TRAIL receptors 1 through 4 was expressed on each cell line, and also indicate the sensitivity or resistance of the cells to TRAIL. TRAIL-R2 mRNA was expressed on all cell lines tested, and TRAIL-R1 mRNA was expressed on most of the cell lines. Some cell lines were positive for TRAIL-R3 and/or TRAIL-R4 mRNA, but fewer than for TRAIL-R1.

A bispecific R1/R2 antibody offers advantages over an antibody that binds only one of the two receptors. If a particular type of cancer cells expresses either of the two receptors, the bispecific antibody will bind to those cells. Use of bispecific antibodies of the invention is particularly advantageous for inducing death of target cells that express both TRAIL-R1 and TRAIL-R2. Methods for determining which of the four TRAIL receptors are expressed in particular cell types are known, with suitable methods including those described in Griffith et al., supra, and Griffith and Lynch, supra.

While not wishing to be bound by theory, regarding mechanism of action for example, bispecific R1/R2 antibodies may promote clustering of apoptosis-inducing receptors. On cells that express both receptors, contacting the cells with bispecific R1/R2 antibodies may result in amassing (or clustering) of both TRAIL-R1 and TRAIL-R2 on the cell surface. In such a scenario, the bispecific antibody may promote amassing of higher concentrations of apoptosis-mediating receptors, compared to what would result from contact with a monospecific antibody (anti-R1 or anti-R2). When an antibody binds a first receptor, the likelihood of a second receptor being physically proximate for binding by a second antigen binding site of the antibody generally will be greater when a bispecific antibody is employed, compared to a monospecific antibody.

Bispecific antibodies may cross-link the two receptors (TRAIL-R1 and TRAIL-R2) on target cells. On cells expressing both receptors, the use of a bispecific R1/R2 antibody (instead of a monospecific antibody) generally increases the incidence of a second receptor being physically close enough to be bound by the antibody.

Another potential advantage of using bispecific antibodies of the invention rather than monospecific antibodies (e.g., a monospecific agonistic TRAIL-R1 MAb) for inducing apoptosis of target cells is as follows. If downregulation or down-modulation of the expression of TRAIL-R1 occurs on target cancer cells during a course of treatment with an anti-R1 MAb, the cancer cells could become resistant to treatment with the TRAIL-R1 MAb. Use of an agonistic bispecific R1/R2 antibody, that induces apoptosis through both TRAIL-R1 and TRAIL-R2, may decrease the likelihood of such resistance developing, since both TRAIL-R1 and TRAIL-R2 would have to be downregulated for the cells to become resistant. Likewise, if receptor turnover alters the number (or type) of receptors present on the surface of target cells during a course of therapy, use of a bispecific antibody that can induce apoptosis through both TRAIL-R1 and TRAIL-R2 could be advantageous.

Numerous methods of preparing bispecific antibodies are known in the art. Bispecific antibodies comprise two different (non-identical) antigen binding sites, such that the antibody specifically binds to two different antigens. Bispecific antibodies of the present invention comprise one antigen-binding region that binds TRAIL-R1, and a second antigen-binding region that binds TRAIL-R2. The two antigen-binding regions are not identical, and bind different epitopes. For preparing agonistic bispecific antibodies, agonistic MAbs that bind TRAIL-R1 or TRAIL-R2 (or hybridomas producing such agonistic MAbs) may be employed as starting materials in various procedures described below.

In any of the procedures described herein for preparing antibodies against TRAIL-R1 or TRAIL-R2, various forms of TRAIL-R1 or TRAIL-R2 may be employed as an immunogen. In particular embodiments, the immunogens are forms of human TRAIL-R1 or TRAIL-R2. Examples of immunogens include, but are not limited to, purified TRAIL-R1 or TRAIL-R2 proteins, immunogenic fragments thereof, fusion proteins thereof such as Fc fusions, or transfected cells expressing high levels of the receptor protein. In one embodiment, a soluble TRAIL-R1 or TRAIL-R2 polypeptide (e.g., the extracellular domain or an immunogenic fragment thereof) is employed as an immunogen.

As an alternative, DNA encoding TRAIL-R1 or TRAIL-R2 can be used as the immunogen. The use of DNA (encoding a desired antigen) as an immunogen is reviewed by Pardoll and Beckerleg in Immunity 3:165, 1995. DNA employed as an immunogen may be given intradermally (Raz et al., Proc. Natl. Acad. Sci. USA 91:9519, 1994) or intamuscularly (Wang et al., Proc. Natl. Acad. Sci. USA 90:4156, 1993); saline has been found to be a suitable diluent for DNA-based immunogens.

One method for preparing bispecific antibodies involves the use of hybridhybridomas as described by Milstein and Cuello (Nature 305:537, 1983). When two hybridoma cells are fused, the resulting cell is referred to as a “quadroma”. In accordance with the present invention, a quadroma cell line is prepared by fusing a hybridoma that secretes a MAb directed against TRAIL-R1 with a hybridoma that secretes a MAb directed against TRAIL-R2. In a particular embodiment, a quadroma cell line is prepared by fusing a hybridoma that secretes an agonistic TRAIL-R1 MAb with a hybridoma that secretes an agonistic TRAIL-R2 MAb. A “trioma” is formed by the fusion of a lymphocyte (derived from an animal that has been immunized with TRAIL-R1) and a hybridoma secreting MAbs that bind TRAIL-R2. Alternatively, a trioma cell line is formed by fusing a lymphocyte from an animal immunized with TRAIL-R2, with a hybridoma secreting a MAb that binds TRAIL-R1. At least a portion of the antibodies produced by hybrid hybridoma cells will be bispecific. For discussion of relevant techniques, see, for example, U.S. Pat. Nos. 4,474,893, 6,106,833, and 5,807,706.

When two hybridomas are chosen for fusion to create a quadroma, both hybridomas advantageously secrete MAbs of the same isotype. In one embodiment, the MAbs both are IgGI antibodies.

One method of the present invention is a method for producing a bispecific R1/R2 antibody. The method comprises fusing hybridoma cells that secrete a monoclonal antibody that binds TRAIL-R1, with hybridoma cells that secrete a monoclonal antibody that binds TRAIL-R2, thereby preparing a hybrid hybridoma that secretes a bispecific R1/R2 monclonal antibody. In one embodiment, the method comprises fusing hybridoma cells that secrete an agonistic TRAIL-R1 MAb, with hybridoma cells that secrete an agonistic TRAIL-R2 MAb. Conventional techniques for conducting such a fusion, and for isolating the desired hybrid hybridoma, include those described elsewhere herein, and those illustrated in the examples below.

U.S. Pat. No. 6,060,285 discloses a process for the production of bispecific antibodies, in which at least the genes for the light chain and the variable portion of the heavy chain of an antibody having a first specificity are transfected into a hybridoma cell secreting an antibody having a second specificity. When the transfected hybridoma cells are cultured, bispecific antibodies are produced, and may be isolated by various means known in the art.

Other investigators have used chemical coupling of antibody fragments to prepare antigen-binding molecules having specificity for two different antigens (Brennan et al., Science 229:81 1985; Glennie et al., J. Immunol. 139:2367, 1987). U.S. Pat. No. 6,010,902 also discusses techniques known in the art by which bispecific antibodies can be prepared, for example by the use of heterobifunctional cross-linking reagents such as GMBS (maleimidobutryloxy succinimide) or SPDP (N-succinimidyl 3-(2-pyridyldithio)propionate). (See, e.g., Hardy, “Purification And Coupling Of Fluorescent Proteins For Use In Flow Cytometry”, Handbook Of Experimental Immunology, 4^{th} Ed., Volume 1, Immunochemistry, Weir et al. (eds.), pp. 31.4-31.12, 1986).

The ability to produce antibodies via recombinant DNA technology has facilitated production of bispecific antibodies. Kostelny et al. utilized the leucine zipper moieties from the fos and jun proteins (which preferentially form heterodimers) to produce bispecific antibodies able to bind both the cell surface molecule CD3 and the receptor for Interleukin-2 (J. Immunol. 148:1547; 1992).

Single chain antibodies may be formed by linking heavy and light chain variable region (Fv region) fragments via an amino acid bridge (short peptide linker), resulting in a single polypeptide chain. Such single-chain Fvs (scFvs) have been prepared by fusing DNA encoding a peptide linker between DNAs encoding the two variable region polypeptides (V_{L} and V_{H}). The resulting antibody fragments can form dimers or higher oligomers, depending on such factors as the length of a flexible linker between the two variable domains (Kortt et al., Protein Engineering 10:423, 1997). In particular embodiments, two or more scFvs are joined by use of a chemical cross-linking agent.

Techniques developed for the production of single chain antibodies can be adapted to produce single chain antibodies of the present invention, that bind both TRAIL-R1 and TRAIL-R2. Such techniques include those described in U.S. Pat. No. 4,946,778; Bird (Science 242:423, 1988); Huston et al. (Proc. Natl. Acad. Sci. USA 85:5879, 1988); and Ward et al. (Nature 334:544, 1989). Once desired single chain antibodies are identified (for example, from a phage-display library), those of skill in the art can further manipulate the DNA encoding the single chain antibody(ies) to yield bispecific antibodies, including bispecific antibodies having Fc regions.

Single chain antibodies against TRAIL-R1 and TRAIL-R2 may be concatamerized in either order (i.e., anti-TRAIL-R1-anti-TRAIL-R2 or anti-TRAIL-R2-anti-TRAIL-R1). In particular embodiments, starting materials for preparing a bispecific R1/R2 antibody include an agonistic single chain antibody directed against TRAIL-R1 and an agonistic single chain antibody directed against TRAIL-R2.

U.S. Pat. No. 5,582,996 discloses the use of complementary interactive domains (such as leucine zipper moieties or other lock and key interactive domain structures) to facilitate heterodimer formation in the production of bispecific antibodies. The complementary interactive domain(s) may be inserted between an Fab fragment and another portion of a heavy chain (i.e., C_{H}1 or C_{H}2 regions of the heavy chain). The use of two different Fab fragments and complementary interactive domains that preferentially heterodimerize will result in bispecific antibody molecules. Cysteine residues may be introduced into the complementary interactive domains to allow disulphide bonding between the complementary interactive domains and stabilize the resulting bispecific antibodies.

Tetravalent, bispecific molecules can be prepared by fusion of DNA encoding the heavy chain of an F(ab′)₂ fragment of an antibody with either DNA encoding the heavy chain of a second F(ab′)₂ molecule (in which the CH1 domain is replaced by a CH3 domain), or with DNA encoding a single chain Fv fragment of an antibody, as described in U.S. Pat. No. 5,959,083. Expression of the resultant fusion genes in mammalian cells, together with the genes for the corresponding light chains, yields tetravalent bispecific molecules having specificity for selected antigens.

Bispecific antibodies can also be produced as described in U.S. Pat. No. 5,807,706, which is incorporated by reference herein. Generally, the method involves introducing a protuberance in a first polypeptide and a corresponding cavity in a second polypeptide, polypeptides interface. The protuberance and cavity are positioned so as to promote heteromultimer formation and hinder homomultimer formation. The protuberance is created by replacing amino acids having small side chains with amino acids having larger side chains. The cavity is created by the opposite approach, i.e., replacing amino acids having relatively large side chains with amino acids having smaller side chains.

The protuberance and cavity can be generated by conventional methods for making amino acid substitutions in polypeptides. For example, a nucleic acid encoding a polypeptide may be altered by conventional in vitro mutagenesis techniques. Alternatively, a polypeptide incorporating a desired amino acid substitution may be prepared by peptide synthesis. Amino acids chosen for substitution are located at the interface between the first and second polypeptides.

For use of antibodies as in vivo diagnostic or therapeutic agents in humans, it is often desirable to use an antibody that is completely or partially human. Many techniques have been developed to facilitate production of such antibodies, examples of which are chimeric or humanized antibodies, or antibodies generated by immunization of transgenic animals, as discussed below. Such an antibody is less likely to generate an immune response than is a completely non-human antibody (e.g., a murine antibody).

Techniques developed for the production of “chimeric” antibodies (i.e., antibodies having portions derived from different species) include those described in Takeda et al. (Nature, 314:452, 1985), Morrison et al. (Proc. Natl. Acad. Sci. USA 81:6851, 1984), Boulianne et al. (Nature, 312:643, 1984), and Neuberger et al. (Nature, 314:268, 1985), for example. One approach to generating chimeric antibodies involves splicing genes from a mouse antibody molecule of appropriate antigen specificity together with genes encoding part or all of the constant region of a human antibody molecule.

A chimeric monoclonal antibody may comprise the variable region of a non-human antibody (or just the antigen binding site thereof) and all or part of the constant region derived from a human antibody. Alternatively, a chimeric antibody can comprise the antigen binding site of a non-human monoclonal antibody and a variable region fragment (lacking the antigen-binding site) derived from a human antibody.

Procedures for the production of engineered monoclonal antibodies that are less likely to generate an immune response in a human include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Such antibodies are referred to as “humanized;” generally, some residues in the hyper-variable or complementarity determining regions (CDRs), and sometimes selected framework residues, in a human antibody are replaced with residues from analogous sites in other (i.e., rodent) antibodies. Useful techniques for humanizing antibodies are also discussed in U.S. Pat. No. 6,054,297.

Such techniques may be employed in preparing humanized bispecific antibodies that bind TRAIL-R1 and TRAIL-R2. For example, chimeric bispecific antibodies may comprise a variable region derived from a murine MAb that binds TRAIL-R1; a second variable region polypeptide, derived from a murine MAb that binds TRAIL-R2; and constant region polypeptides derived from a human antibody.

Other techniques for generating partially or completely human antibodies involve the use of transgenic animals, in which human immunoglobulin polypeptide(s) are expressed in place of endogenous immunoglobulin polypeptide(s). Examples of such transgenic animals are mice in which endogenous immunoglobulin genes (particularly heavy chain genes) are replaced by human immunoglobulin genes. Examples of techniques for production and use of such transgenic animals are described in U.S. Pat. Nos. 5,814,318, 5,569,825, and 5,545,806, and GB 2,272,440, which are incorporated by reference herein.

Mice may be genetically altered in a variety of ways, to create transgenic mice useful for producing human antibodies. Techniques are known for producing mice in which one or more endogenous immunoglobulin genes have been inactivated by various means. Human immunoglobulin genes are introduced into the mice to replace the inactivated mouse genes. Antibodies produced in the animals incorporate the human immunoglobulin polypeptide chains encoded by the human genetic material that was introduced into the animal. The genetic manipulation results in human immunoglobulin polypeptide chains replacing endogenous immunoglobulin chains in at least some (preferably virtually all) antibodies produced by the animal upon immunization. The antibodies may be partially human, or preferably completely human.

Antibodies produced by procedures that comprise immunizing transgenic animals with a TRAIL-R1 or TRAIL-R2 polypeptide may be employed in preparing bispecific antibodies. Transgenic mice into which genetic material encoding human immunoglobulin polypeptide chain(s) has been introduced are among the suitable transgenic animals.

One method for producing a hybridoma cell line comprises immunizing such a transgenic animal with a TRAIL-R1 or TRAIL-R2 immunogen; harvesting spleen cells from the immunized animal; fusing the harvested spleen cells to a myeloma cell line, thereby generating hybridoma cells; and identifying a hybridoma cell line that produces a monoclonal antibody that binds TRAIL-R1 (or TRAIL-R2). Quadromas or triomas may be derived from the hybridomas secreting human MAbs, using procedures described above. Bispecific R1/R2 MAbs that are partially or fully human thus are prepared.

The desired bispecific antibodies can be identified and isolated by utilizing affinity chromatography with a first TRAIL receptor (TRAIL-R1), then using a second affinity chromatography step wherein the second TRAIL receptor (TRAIL-R2) is used as the binding moiety. Antibodies that bind only the first TRAIL receptor will flow through the second affinity column, while antibodies that also bind the second TRAIL receptor will bind to the column matrix, and be eluted under the appropriate conditions.

Cells that produce bispecific R1/R2 antibodies are encompassed by the present invention. Such cells include, but are not limited to, quadroma or trioma cell lines that secrete bispecific anti-R1/R2 monoclonal antibodies, as discussed above.

Certain bispecific R1/R2 antibodies may function as blockers, in that the antibody is capable of inhibiting a biological effect that results from binding of TRAIL to cell surface TRAIL-R1 or TRAIL-R2. Antibodies that inhibit one or more biological activities of TRAIL may be employed as TRAIL antagonists. Any of a number of conventional assays may be employed to identify bispecific R1/R2 antibodies that function as TRAIL antagonists. An antibody may be tested for the ability to inhibit binding of TRAIL to cells. One alternative involves testing an antibody for the ability to inhibit TRAIL-induced apoptosis of TRAIL-sensitive target cells, such as Jurkat cells.

As discussed above, other bispecific R1/R2 antibodies provided herein are agonistic. Agonistic bispecific R1/R2 antibodies mimic a biological activity of the cognate ligand (TRAIL), e.g., the antibodies are capable of inducing apoptosis of transformed target cells. TRAIL has been reported to induce death of a number of different types of cancer cells. Cancer cell types that are sensitive to TRAIL include, but are not limited to, those discussed in Wiley et al. (Immunity 3:673-682, 1995), Griffith and Lynch (Current Opinion in Immunology, 10:559-563, 1998), Walczak et al. (Nature Medicine 5:157, 1999), Griffith et al. (J. Immunol. 162:2597, 1999), and U.S. Pat. No. 5,763,223. It is expected that cells that are killed by contact with TRAIL express at least one of the two apoptosis-mediating receptors (TRAIL-R1 or TRAIL-R2). TRAIL-sensitive cancer cells are among the types of target cells that may be killed by contact with an agonistic bispecific R1/R2 antibody of the present invention. Virally-infected cells are another example of target cells that may be killed by contact with agonistic bispecific R1/R2 antibodies (see U.S. Pat. No. 5,763,223).

Bispecific antibodies that bind TRAIL-R1 and TRAIL-R2 may be screened for agonistic (ligand-mimicking) properties, by using any of a number of conventional techniques. Cell viability assays and apoptosis assays are among the types of assays that may be employed to identify bispecific R1/R2 antibodies that are capable of killing target cells. Among the suitable techniques are those described in Wiley et al. (Immunity 3:673-682, 1995) and in U.S. Pat. No. 5,763,223, for demonstrating the ability of TRAIL to kill target cells. Other suitable assays are described in examples 5 and 6 below.

A characteristic DNA laddering pattern is recognized as a hallmark of apoptotic cell death. Techniques for visualizing such DNA fragmentation are known. Certain of the techniques involve resolving the fragmented DNA by agarose gel electrophoresis, and the use of dyes that allow visualization of DNA.

One way to confirm cell death is by staining the target cells with trypan blue. An alternative is crystal violet staining, performed as described by Flick and Gifford (J. Immunol. Methods 68:167-175, 1984).

Embodiments of antibodies provided herein are bispecific antibodies that induce an apoptotic signal through TRAIL-R1 or TRAIL-R2, upon binding to a cell. Preferred bispecific antibodies induce an apoptotic signal through both TRAIL-R1 and TRAIL-R2. In one approach, such a preferred antibody is derived from two MAbs, an agonistic TRAIL-R1 MAb and an agonistic TRAIL-R2 MAb. Suitable parent antibodies (agonistic R1 and agonistic R2 MAbs) may be identified by techniques such as those described above, which identify agonistic antibodies that induce death of target cells. Bispecific R1/R2 MAbs, produced from the monospecific parent MAbs, also may be tested in such assays to confirm the ability to kill target cells.

A bispecific antibody's ability to induce cell death through both TRAIL-R1 and TRAIL-R2 may be confirmed by any of a number of conventional techniques. For example, target cells may be contacted with an antagonistic (blocking) antibody directed against TRAIL-R1, then contacted with a bispecific antibody. Target cell death is determined. In a separate assay, the target cells are contacted with an antagonistic (blocking) antibody directed against TRAIL-R2, then contacted with a bispecific antibody, and target cell death is determined. Bispecific antibodies that induce target cell death through both TRAIL-R1 and TRAIL-R2 thus are identified.

In particular embodiments, parent and/or bispecific antibodies may be screened for additional desired properties. For example, binding affinity for the receptors may be determined by conventional techniques. In one embodiment, a bispecific R1/R2 MAb exhibits comparable binding affinity for both TRAIL-R1 and TRAIL-R2.

One method provided herein is a method for killing cancer cells, comprising contacting cancer cells with an agonistic bispecific antibody that binds TRAIL-R1 and TRAIL-R2. Another method provided herein is a method for killing virally infected cells, comprising contacting virally infected cells with an agonistic bispecific antibody that binds TRAIL-R1 and TRAIL-R2. Agonistic bispecific R1/R2 antibodies may be employed to kill target cells in procedures in which the antibody contacts the target cells in vitro, in vivo, or ex vivo. In methods provided herein that comprise administration of a bispecific R1/R2 antibody, it is to be understood that such methods encompass administration of one or more different bispecific R1/R2 antibodies.

Whole antibodies, comprising Fc regions, are generally preferred for in vivo administration, to kill cancer cells or virally infected cells in the methods provided herein. Bispecific antibodies that comprise at least one Fc region polypeptide are provided. Genetic engineering or protein engineering techniques may be employed to prepare a bispecific antibody with two antigen binding regions (one of which is immunoreactive with TRAIL-R1, the other immunoreactive with TRAIL-R2), and one or two Fc region polypeptides, for example.

An agonistic antibody directed against TRAIL-R1 may be used in combination with an agonistic antibody directed against TRAIL-R2 (i.e., two monospecific antibodies) to kill cancer cells or virally infected cells, in the methods disclosed herein. However, bispecific R1/R2 antibodies are preferred for such use.

A method for killing cancer cells in vivo comprises administering an agonistic bispecific R1/R2 antibody to a mammal, preferably a human, who has been diagnosed with cancer. The present invention provides a method for treating a mammal, preferably a human, who has cancer, comprising administering to the mammal a bispecific antibody that binds TRAIL-R1 and TRAIL-R2, wherein the antibody is capable of killing cancer cells. The antibody preferably is a monoclonal antibody. When the patient is a human, the bispecific antibody advantageously binds to human TRAIL-R1 and human TRAIL-R2. Individuals who may be treated according to the present invention include, for example, those afflicted with any neoplastic condition characterized by cells that express TRAIL-R1 or TRAIL-R2, advantageously both TRAIL-R1 and TRAIL-R2. Methods provided herein may be employed to achieve such therapeutic objectives as a reduction of tumor burden in a mammal.

Examples of types of cancer that may be treated include, but are not limited to, carcinomas, sarcomas, lymphomas, leukemia, melanoma, multiple myeloma, cancers of the lung, breast, ovary, cervix, prostate, kidney, liver, bladder, pancreas, stomach, colon (including colorectal cancer), skin, and nervous system. Particular examples include, but are not limited to, colon carcinoma, carcinoma of the breast, small-cell lung cancer, and non-small-cell lung cancer. Conventional techniques may be employed to confirm the susceptibility of various types of cancer cells to cell death induced by bispecific antibodies of the present invention.

An agonistic bispecific R1/R2 antibody may administered alone, or may be co-administered with one or more additional agents that are useful in treating cancer. Coadministration is not limited to simultaneous administration, but includes treatment regimens in which such an antibody is administered at least once during a course of treatment that involves administering at least one other agent to the patient.

In one method of the invention, the bispecific R1/R2 antibody is administered to the patient prior to administration of a second anti-cancer agent. One alternative method comprises administering the second anti-cancer agent prior to administering the bispecific antibody. Particular methods may involve administering the bispecific antibody and second agent on an alternating schedule. In another embodiment, the bispecific antibody and second agent are administered simultaneously.

Examples of such agents include both proteinaceous and non-proteinaceous drugs, and radiation therapy. The choice of such agents will vary according to such factors as the type of cancer and condition of the patient. Examples of proteinaceous agents include various cytokines that induce a desired immune or other biological response, interferons such as γ-interferon, TRAIL, and other antibodies. TRAIL is described in U.S. Pat. No. 5,763,223 (hereby incorporated by reference) One example of an antibody employed in cancer treatment is Herceptin® (Genentech, South San Francisco, Calif.).

A wide variety of drugs have been employed in chemotherapy of cancer. Examples include, but are not limited to, cisplatin, taxol, etoposide, Novantrone® (mitoxantrone), actinomycin D, camptothecin (or water soluble derivatives thereof such as irinotecan or topotecan), methotrexate, gemcitabine, mitomycin (e.g., mitomycin C), dacarbazine (DTIC), 5-fluorouracil, and anti-neoplastic antibiotics such as doxorubicin and daunomycin.

Examples of particular combinations of drugs with bispecific antibodies, that may be co-administered in accordance with the present invention, include but are not limited to the following. Particular embodiments of methods of the invention comprise co-administering an agonistic bispecific R1/R2 MAb with methotrexate, etoposide, or mitoxantrone to a cancer patient, including but not limited to prostate cancer patients. A method for treating colorectal cancer or colon cancer, such as colon carcinoma, comprises co-administering an agonistic bispecific R1/R2 MAb with a water soluble derivative of camptothecin, such as topotecan or, preferably, irinotecan (CPT-11). A method for treating melanoma comprises co-adrninistering a bispecific R1/R2 MAb with actinomycin D or cyclohexamide.

Drugs employed in cancer therapy may have a cytotoxic or cytostatic effect on cancer cells, or may reduce proliferation of the malignant cells. Among the texts providing guidance for cancer therapy is Cancer, Principles and Practice of Oncology, 4th Edition, DeVita et al., Eds. J. B. Lippincott Co., Philadelphia, Pa. (1993). An appropriate therapeutic approach is chosen according to such factors as the particular type of cancer and the general condition of the patient, as is recognized in the pertinent field.

In one approach, an agonistic bispecific R1/R2 MAb is added to a standard chemotherapy regimen, in treating a cancer patient. For those combinations in which the antibody and additional anti-cancer agent(s) exert a synergistic effect against cancer cells, the dosage of the additional agent(s) may be reduced, compared to the standard dosage of the second agent when administered alone. The antibody may be co-administered with an amount of an anti-cancer drug that is effective in enhancing sensitivity of cancer cells to the antibody.

Agonistic bispecific antibodies that bind TRAIL-R1 and TRAIL-R2 may be employed in treating viral infections and associated conditions arising from viral infections. A method for treating an individual afflicted with a disease or condition caused by a virus that is sensitive to TRAIL, comprises administering to the individual a bispecific antibody that binds TRAIL-R1 and TRAIL-R2, wherein the antibody is capable of killing virally-infected cells.

A method for killing virally infected cells in vivo comprises administering an agonistic bispecific R1/R2 antibody to a mammal, preferably a human, who is infected with a virus. Viral infections include, but are not limited to, infection with cytomegalovirus, influenza, Newcastle disease virus, vesicular stomatitus virus, herpes simplex virus, hepatitis, adenovirus-2, bovine viral diarrhea virus, human immunodeficiency virus (HIV), and Epstein-Barr virus. Encephalomyocarditis is another example of a viral disease that may be treated with an agonistic bispecific antibody. Cells infected with a particular virus may be tested for expression of TRAIL-R1 and/or TRAIL-R2 by conventional techniques, such as techniques analogous to those described above for testing cancer cells for expression of TRAIL receptor niRNA or cell surface protein. Alternatively, conventional techniques may be employed to confirm the susceptibility of cells infected with various types of viruses, to cell death induced by agonistic bispecific antibodies of the present invention.

Bispecific antibodies of the present invention may be administered alone or in combination with one or more additional anti-viral agent(s) useful for combating a particular virus. As one example, a bispecific R1/R2 monoclonal antibody is co-administered with an interferon, e.g., γ-interferon, to treat a viral infection.

In one method of the invention, the bispecific R1/R2 antibody is administered to the patient prior to administration of a second anti-viral agent. One alternative method comprises administering the second anti-viral agent prior to administering the bispecific antibody. Particular methods may involve administering the bispecific antibody and second agent on an alternating schedule. In another embodiment, the bispecific antibody and second agent are administered simultaneously.

A bispecific antibody may be co-administered with one or more agents that inhibit viral replication. In a particular embodiment, the virus is human immunodeficiency virus (HIV) and the antibody is co-administered with at least one anti-retroviral agent. The antiretroviral agent may be any pharmacological, biological or cellular agent that has demonstrated the ability to inhibit HIV replication.

One therapeutic approach comprises treating an HIV⁺ human by co-administering the antibody with at least one (preferably at least two) drugs selected from protease inhibitors, nucleoside analogs that inhibit reverse transcriptase, and non-nucleoside reverse transcriptase inhibitors. One approach involves co-administering a bispecific antibody with a drug cocktail comprising three anti-retroviral agents, including more than one class of antiretroviral agent. In one method provided herein, the drug cocktail comprises a protease inhibitor and at least one (preferably two) nucleoside reverse transcriptase inhibitors.

Examples of antiretroviral agents that may be employed in methods of the present invention, include, but are not limited to, nucleoside reverse transcriptase inhibitors, nonnucleoside reverse transcriptase inhibitors, protease inhibitors. Specific examples of nucleoside reverse transcriptase inhibitors include zidovudine (AZT), didanosine (ddI), lamivudine (3TC), stavudine (d4T), and dalcitabine (ddC). Specific examples of nonnucleoside reverse transcriptase inhibitors include nevirapine and delavirdine. Specific examples of protease inhibitors include indinavir, nelfinavir, ritonavir, and saquinavir. Further examples of anti-HIV drugs are HIV integrase inhibitors and agents that block viral entry through chemokine receptors. Examples of chemokine receptor blocking agents are small peptides known as CXCR4 or CCR4 blocking peptides.

Compositions comprising an effective amount of a bispecific antibody of the present invention, in combination with other components such as a physiologically acceptable diluent, carrier, or excipient, are provided herein. The antibody can be formulated according to known methods used to prepare pharmaceutically useful compositions. An antibody can be combined in admixture, either as the sole active material or with other known active materials suitable for a given indication, with pharmaceutically acceptable diluents (e.g., saline, Tris-HCl, acetate, or phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa.

In addition, such compositions can contain an antibody attached to polyethylene glycol (PEG), or metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of the antibody, and are thus chosen according to the intended application.

Compositions of the present invention may contain an antibody in any form described herein. In one embodiment, a composition comprises an antigen-binding fragment of a bispecific antibody (wherein the antibody fragment binds both TRAIL-R1 and TRAIL-R2) together with a physiologically acceptable diluent, carrier, or excipient. Preferably, the composition comprises a bispecific antibody that comprises at least one Fc region polypeptide (one embodiment of which is a whole antibody).

Bispecific antibodies provided herein may be administered in any suitable manner, e.g., topically, parenterally, or by inhalation. The term “parenteral” includes injection, e.g., by subcutaneous, intravenous, or intramuscular routes, also including localized administration, e.g., at a site of disease or injury. Sustained release from implants is also contemplated. One skilled in the pertinent art will recognize that suitable dosages will vary, depending upon such factors as the nature of the disorder to be treated, the patient's body weight, age, and general condition, and the route of administration. Preliminary doses can be determined according to animal tests, and the scaling of dosages for human administration are performed according to art-accepted practices.

Bispecific antibodies provided herein also find use as carriers for delivering agents attached thereto to cells bearing TRAIL-R1 and/or TRAIL-R2, such as cancer cells expressing the receptor(s), for example. The antibodies can be used to deliver diagnostic or therapeutic agents to such cells in in vitro, ex vivo, or in vivo procedures. Conjugates comprising a diagnostic (detectable) or therapeutic agent and a bispecific antibody of the invention are provided herein.

Therapeutic agents that may be attached to an antibody include, but are not limited to, toxins, other cytotoxic agents, drugs, radionuclides, and the like, with the particular agent being chosen according to the intended application. Among the toxins are ricin, abrin, diphtheria toxin, Pseudomonas aeruginosa exotoxin A, ribosomal inactivating proteins, mycotoxins such as trichothecenes, and derivatives and fragments (e.g., single chains) thereof. In particular embodiments, the drug is in a precursor form that is processed to active form in vivo, e.g., after being internalized into a cell. Detectable (diagnostic) agents that may be attached to an antibody include, but are not limited to, radionuclides, chromophores, and enzymes that catalyze a calorimetric or fluorometric reaction. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu.

Such agents may be attached to the antibody by any suitable conventional procedure. Antibodies comprise functional groups on amino acid side chains that can be reacted with functional groups on a desired agent to form covalent bonds, for example. Alternatively, the antibody or agent may be derivatized to generate or attach a desired reactive functional group. The derivatization may involve attachment of one of the bifunctional coupling reagents available for attaching various molecules to proteins (such as those avalable from Pierce Chemical Company, Rockford, Ill.). A number of techniques for radiolabeling antibodies are known. Radionuclide metals may be attached to a bispecific antibody by using a suitable bifunctional chelating agent, for example.

The following examples are offered by way of illustration, and not by way of limitation. Those skilled in the art will recognize that variations of the invention embodied in the examples can be made, especially in light of the teachings of the various references cited herein, the disclosures of which are incorporated by reference.

### EXAMPLE 1

### Monoclonal Antibodies directed against TRAIL-R1 or TRAIL-R2

This example illustrates the preparation of hybridoma cell lines secreting monoclonal antibodies (MAbs) that bind TRAIL-R1, and hybridomas secreting MAbs that bind TRAIL-R2. The hybridomas are employed as starting materials in preparing bispecific MAbs, as described in example 2.

Monoclonal antibodies may be prepared by conventional techniques. See, for example, Monoclonal Antibodies, Hybridomas: A New Dimension in Biological Analyses, Kennet et al. (eds.), Plenum Press, New York (1980); Antibodies: A Laboratory Manual, Harlow and Land (eds.), Cold Spring Harbor Laboratory Press, Cold Spring Harbor, N.Y., (1988); and the techniques disclosed in U.S. Pat. No. 4,411,993.

Purified TRAIL-R1 or TRAIL-R2 protein, or an immunogenic fragment thereof, may be employed as the immunogen. In one embodiment, a soluble fragment of TRAIL-R1 or TRAIL-R2 (e.g., the extracellular domain or an immunogenic fragment thereof) is employed as an immunogen.

To immunize rodents, a proteinaceous TRAIL-R1 immunogen is emulsified in an adjuvant (such as complete or incomplete Freund's adjuvant, alum, or Ribi adjuvant R700 (Ribi, Hamilton, Mont.)), and injected in amounts ranging from 10-100 μg subcutaneously into a selected rodent, for example, BALB/c mice or Lewis rats. Ten days to three weeks later, the immunized animals are boosted with additional immunogen emulsified in adjuvant, and periodically boosted thereafter on a weekly, biweekly or every third week immunization schedule.

Serum samples are periodically taken by retro-orbital bleeding or tail-tip excision, to test for antibodies against TRAIL-R1. The testing involves dot-blot assay (antibody sandwich), ELISA (enzyme-linked immunosorbent assay), immunoprecipitation, or other suitable assays, including FACS analysis. Following detection of an appropriate antibody titer, positive animals are given an intravenous injection of immunogen in saline. Three to four days later, the animals are sacrificed, splenocytes harvested, and the splenocytes are fused to a murine myeloma cell line (e.g., NS1 or preferably P3×63Ag8.653 (ATCC CRL 1580)). Hybridoma cells generated by this procedure are plated in multiple microtiter plates in a selective medium, such as growth medium containing hypoxanthine, aminopterin, and thymidine (HAT), to inhibit proliferation of non-fused cells, myelomamyeloma hybrids, and splenocyte-splenocyte hybrids.

Hybridoma clones thus generated can be screened by ELISA for reactivity with TRAIL-R1, by adaptations of the techniques disclosed by Engvall et al., Immunochem. 8:871 (1971) and in U.S. Pat. No. 4,703,004, for example. One screening technique is the antibody capture technique described by Beckman et al., J. Immunol. 144:4212 (1990). One of the suitable assay procedures is illustrated in example 3 (section a) below.

Hybridoma clones that test positive in such assays are then injected into the peritoneal cavities of syngeneic rodents, to produce ascites containing high concentrations (>1 mg/ml) of TRAIL-R1 MAb. The monoclonal antibodies can be purified by ammonium sulfate precipitation followed by gel exclusion chromatography. Alternatively, affinity chromatography based upon binding of antibody to protein A or protein G can also be used, as can affinity chromatography based upon binding to TRAIL-R1. The MAbs are screened to confirm reactivity against TRAIL-R1.

Hybridoma cells lines secreting monoclonal antibodies that bind TRAIL-R2 are generated by using the same procedure, but employing TRAIL-R2 as the immunogen. The monoclonal antibodies are purified and screened to confirm reactivity against TRAIL-R2, using procedures discussed above.

### EXAMPLE 2

### Bispecific Antibodies

Bispecific antibodies that bind both TRAIL-R1 and TRAIL-R2 may be prepared as follows. The bispecific antibodies may be derived from hybridoma cell lines prepared as described in Example 1. Other procedures employ lymphocytes from mice immunized with TRAIL-R1 or TRAIL-R2, as described in Example 1.

In one approach, quadroma cell lines expressing bispecific antibodies are obtained by fusing two hybridoma cell lines, wherein one of the hybridomas secretes MAbs that bind TRAIL-R1, and the other secretes MAbs that bind TRAIL-R2. The fusion to create the quadroma is conducted under conditions substantially similar to those described above for generation of the original hybridomas.

In another approach, trioma cell lines are obtained by fusing a hybridoma cell line secreting monoclonal antibodies having specificity for TRAIL-R1 with lymphocytes extracted from a mouse that has been immunized with TRAIL-R2. Alternatively, a trioma is prepared by fusing a hybridoma secreting MAbs against TRAIL-R2 with lymphocytes from a mouse that has been immunized with TRAIL-R1.

Cell lines secreting bispecific antibodies can also be obtained by simultaneous three-way fusion. For example, lymphocytes from animals immunized with TRAIL-R1, and lymphocytes from animals immunized with TRAIL-R2 are mixed together with a suitable fusion partner (e.g., myeloma cell lines as described in example 1). Alternatively, a single mouse (for example, a transgenic mouse having at least some human immunoglobulin genes) can be immunized with both TRAIL receptors; lymphocytes obtained in this manner can then be fused to suitable immortalized cells using the above-described techniques.

Regardless of the method used to obtain cells secreting bispecific antibodies, such cells can be identified by routine procedures, using one or more of the assays described below, then cloned and subcloned to develop a stable, antibody-secreting cell line for standard hybridoma cells. The cell lines can then be used in any technique known in the art (for example, growth in the peritoneal cavity of mice or large-scale culture) to obtain large quantities of bispecific antibodies.

### EXAMPLE 3

### Binding Assays

This example describes three solid-phase binding assays which can be used to detect, quantitate or characterize antibodies that bind TRAIL receptors.

(a) Quantitative TRAIL receptor antibody-specific ELISA

A TRAIL receptor protein (or a fusion protein thereof) is prepared and purified by methods that are known in the art, and used to coat 96-well plates (Corning EasyWash ELISA plates, Corning, N.Y., USA). The plates are coated with from about 1.5 to 3.5 μg/well of the protein in PBS overnight at 4° C., and blocked with 1% non-fat milk in PBS for 1 hour at room temperature. Samples to be tested are diluted in 10% normal goat serum in PBS, and 50 μl is added per well. A titration of unknown samples is run in duplicate, and a titration of reference standard of TRAIL receptor antibody may be run to generate a standard curve.

The plates are incubated with the samples and controls for from 30 to 60 minutes at room temperature, then washed about four times with PBS. Second step reagent, for example, rabbit anti-murine immunoglobulin, is added (50 μl/well, concentration approximately 2.5 μg/ml), and the plates are incubated at room temperature for from 30 to 60 minutes. The plates are again washed as previously described, and goat F(ab′)2 anti-rabbit IgG conjugated to horseradish peroxidase (Tago, Burlingame, Calif., USA) is added. Plates are incubated for 45 minutes at room temperature, washed as described, and the presence of TRAIL receptor antibodies is detected by the addition of chromogen, tetramethyl benzidene (TMB; 100 μl/well) for 15 minutes at room temperature. The chromogenic reaction is stopped by the addition of 100 μl/well 2N H₂SO₄, and the OD₄₅₀-OD₅₆₂ of the wells determined. The quantity of TRAIL receptor antibodies can be determined by comparing the OD values obtained with the unknown samples to the values generated for the standard curve. Those of skill in the art will recognize that the parameters of the above-described ELISA can be optimized or varied to facilitate detection of TRAIL receptor antibodies. When screening for bispecific antibodies, samples of fluid containing putative antibodies are assayed in separate ELISAs utilizing either TRAIL receptor 1 or TRAIL receptor 2. Those samples that react with both TRAIL receptors are further analyzed to identify cells secreting bispecific antibodies.

The reagents employed in the assay are chosen according to the antibody to the tested. For example, if the anti-TRAIL receptor antibody is a human antibody prepared by immunizing transgenic mice, the second step reagent advantageously should be an anti-human immunoglobulin.

(b) Single ELISA

A first TRAIL receptor protein (or a fusion protein thereof) is prepared and purified by methods that are known in the art, and used to coat 96-well plates (Coming EasyWash ELISA plates, Coming, N.Y., USA), substantially as described previously. The plates are incubated with the samples and controls for from 30 to 60 minutes at room temperature, then washed about four times with PBS.

Second step reagent, consisting of the second TRAIL receptor protein conjugated to biotin (for example), is added (50 μl/well, concentration approximately 2.5 μg/ml), and the plates are incubated at room temperature for from 30 to 60 minutes. The plates are again washed as previously described, and a detecting reagent (i.e., streptavidinconjugated horseradish peroxidase) is added. Plates are incubated for 45 minutes at room temperature, washed as described, and the presence of bispecific TRAIL receptor antibodies is detected by the addition of chromogen, tetramethyl benzidene (TMB; 100 μl/well) for 15 minutes at room temperature. The chromogenic reaction is stopped by the addition of 100 μl/well 2N H₂SO₄, and the OD₄₅₀-OD₅₆₂ of the wells determined. Those of skill in the art will recognize that the parameters of the above-described ELISA can be optimized or varied to facilitate detection of TRAIL receptor antibodies.

(c) Affinity Determination Using a Biosensor

This example illustrates a method to determine or compare the binding affinities of TRAIL receptor antibodies. Affinity experiments are conducted by biospecific interaction analysis (BIA) using a biosensor, an instrument that combines a biological recognition mechanism with a sensing device or transducer. An exemplary biosensor is BIAcore™, from Pharmacia Biosensor AB (Uppsala, Sweden; see Fägerstam L. G., Techniques in Protein Chemistry II, ed. J. J. Villafranca, Acad. Press, N.Y., 1991). BIAcore™ uses the optical phenomenon surface plasmon resonance (Kretschmann and Raether, Z. Naturforschung, Teil. A 23:2135, 1968) to monitor the interaction of two biological molecules. Molecule pairs having affinity constants in the range of 10⁵ to 10¹⁰ M⁻¹, and association rate constants in the range of 10³ to 10⁶ M⁻¹ s⁻¹, are suitable for characterization with BIAcore™.

The biosensor chips are coated with a TRAIL receptor protein, by directly or indirectly binding TRAIL receptor to the chip. Methods whereby TRAIL receptor is indirectly bound involve, for example, first coating the chip with an antibody directed against an Fc polypeptide, which then binds a TRAIL receptor/Fc fusion protein. In alternative procedures, an antibody that binds a tag protein such as FLAG® or poly-His is attached to the chip, and subsequently binds the corresponding tag-TRAIL receptor fusion protein.

Antibodies to be tested for the ability to bind the TRAIL receptor are then added at increasing concentrations. The chip is regenerated between the different antibodies by the addition of sodium hydroxide. The resultant data can be analyzed to determine the affinity and association rate constants of the various TRAIL receptor antibodies. With bispecific antibodies, the affinity and association rate constants for each TRAIL receptor can be determined; moreover, such assays will be useful in selecting antibodies with desired affinity and/or association rates to use in preparing bispecific antibodies.

### EXAMPLE 4

### Purification of Bispecific antibodies

This example describes a method of purifying bispecific antibodies that bind TRAIL-R1 and TRAIL-R2. Once cells expressing bispecific antibodies are identified, large scale cultures of cells are grown to accumulate supernatant from cells expressing bispecific antibodies, or the cells are injected into the peritoneal cavities of mice to yield ascitic fluid containing bispecific antibodies. The resulting bispecific antibodies are purified by affinity purification. Briefly, culture supernatant or acites containing bispecific antibodies is filtered (e.g., using a 0.45 micron filter) and the filtrate is applied to an affinity column in which the binding moiety is a first TRAIL receptor. Conditions for binding are determined by routine experimentation, and will usually be at 4° C. at a flow rate of 80 ml/hr for a 1.5 cm×12.0 cm column. The column is washed with suitable wash buffer until free protein is not detected in the wash buffer. Bound antibody is eluted from the column, for example, by using a low pH buffer or any other suitable method of disrupting the antigen-antibody complex known in the art.

The eluate will contain both bispecific antibodies and monospecific antibodies that bind the first TRAIL receptor. The monospecific antibodies are removed by performing a second affinity purification step in which the affinity binding moiety present in the column is the second TRAIL receptor. The eluate from the first column is applied to the second affinity column under conditions promoting binding to the second TRAIL receptor, and the column is washed until free protein can not be detected. The antibodies that remain bound to the second column will thus be bispecific antibodies, and can be eluted by methods similar to those used in the first column. Silver-stained SDS gels of the eluted bispecific antibodies can be performed to determine percent purity. The purified bispecific antibodies can also be evaluated by any quantitative or qualitative assay described herein, and utilized in vitro or in vivo to evaluate their effects on cells expressing TRAIL-R1 and/or TRAIL-R2.

### EXAMPLE 5

### Biologic Effects

This example describes methods of evaluating the cytotoxic effect of bispecific antibodies that bind TRAIL-R1 and TRAIL-R2 on cancer cells. A bispecific antibody may be assayed for anti-tumor activity, using any of a number of suitable assays, including but not limited to assays for the ability to slow tumor growth or to shrink established tumors in vivo, or to kill cancer cells in vitro. Various tumor-derived cell lines are among the target cells that may be contacted with a bispecific antibody, in such assay procedures.

Numerous methods of evaluating the in vivo effects of anti-tumor agents are known in the art. One method involves injecting human tumor cells into mice, and testing the effect of the anti-tumor agent on growth of the tumor cells in the mice, e.g., as described in Walczak et al. (Nature Medicine 5:157-163, 1999). Briefly, the human mammary adenocarcinoma cell line MDA-231 (or other suitable TRAIL receptor positive tumor cell line) is injected into host animals, such as CB.17 (SCID) mice. Bispecific R1/R2 MAb or control reagent is administered at selected time points during the course of tumor development. The effect of the bispecific R1/R2 MAb, compared to the control, is determined by evaluating tumor development in the host animal (e.g. monitoring tumor size), or by histologic examination of tumor tissue extracted from the mice after treatment with the antibody.

For in vitro assays, cell lines are cultured in suitable growth medium, such as DMEM supplemented with 10% fetal bovine serum, penicillin, streptomycin and glutamine. The cells are incubated (e.g., in 96-well culture plates) with the antibody to be tested either in solution or immobilized to the culture plate. In one approach, the bispecific MAb is crosslinked by using a MAb specific for the Fc region. Cell death is determined by any of the many techniques for assessing cell viability, e.g., by chromium release (⁵¹Cr-release) assay (after 8 hours of incubation with the antibody) or crystal violet staining (after 24 hours incubation with the antibody). Detailed procedures for examples of the many suitable assays are as follows.

DNA Laddering Apoptosis Assay

COLO-205 cells, a human colorectal cancer (specifically colon adenocarcinoma) cell line, are used as the target cells in this assay. COLO-205 is available from the American Type Culture Collection, Manassas, Virginia, as ATCC CCL-222.

The COLO-205 cells are cultured under conventional conditions, to a density of 200,000 to 500,000 cells per ml. Four million of these cells per well are co-cultured in a 6-well plate with 2.5 mls of media and the test antibody or control. The plates are coated with antibody at 10 μg/ml.

After four hours the cells are washed once in PBS and pelleted at 1200 rpm for 5 minutes in a desktop centrifuge. The pellets are resuspended and incubated for ten minutes at 4° C. in 500 μl of buffer consisting of 10 mM Tris-HCl, 10 mM EDTA, pH 7.5, and 0.2% Triton X-100, which lyses the cells but leaves the nuclei intact. The lysate was then spun at 4° C. for ten minutes in a micro-centrifuge at 14,000 rpm. The supernatants are removed and extracted three times with 1 ml of 25:24:1 phenol-chloroform-isoamyl alcohol, followed by precipitation with NaOAC and ethanol in the presence of 1 μg of glycogen carrier (Sigma).

The resulting pellets are resuspended in 10 mM Tris-HCl, 10 mM EDTA, pH 7.5, and incubated with 10 μg/ml RNase A at 37° C. for 20 minutes. The DNA solutions are then resolved by 1.5% agarose gel electrophoresis in Tris-Borate EDTA buffer. The gel then is stained with ethidium bromide and photographed while trans-illuminated with UV light, to visualize DNA laddering. Fragmentation of cellular DNA into a pattern known as DNA laddering is a hallmark of apoptosis.

Alamar Blue Conversion Assay

This assay can be used to demonstrate the ability of an agonistic antibody of the invention to cause a significant reduction in viability of COLO-205 cells (or other cancer cells) compared to control. Cancer cells are cultured under conventional conditions, to a density of 200,000 to 500,000 cells per ml. The cells (in 96-well plates at 50,000 cells per well in a volume of 100 μl) are incubated for twenty hours with test antibody or control.

Metabolic activity of the thus-treated cells is assayed by metabolic conversion of alamar Blue dye, in the following procedure. Alamar Blue conversion is measured by adding 10 μl of alamar Blue dye (Biosource International, Camarillo, Calif.) per well, and subtracting the optical density (OD) at 550-600 nm at the time the dye is added from the OD 550-600 nm after four hours. No conversion of dye is plotted as 0 percent viability, and the level of dye conversion in the absence of the test antibody is plotted as 100 percent viability. Percent viability is calculated by multiplying the ratio of staining of experimental versus control cultures by 100.

Crystal Violet Staining Assay

For adherent cell lines, a crystal violet assay, rather than alamar Blue, is prefered for determining cell viability. Target cells are cultured in DMEM supplemented with 10% fetal bovine serum, 100 μg/ml streptomycin, and 100 μg/ml penicillin. The cells (in 96-well plates at 10,000 cells per well in a volume of 100 μl) are incubated for 72 hours with the antibody of interest. Crystal violet staining is performed as described by (Flick and Gifford (J. Immunol. Methods 68:167-175, 1984).

Target cells

Other types of cancer cells may be employed as target cells in any of the above-described in vitro assays. For testing bispecific antibodies, the target cells advantageously express both TRAIL-R1 and TRAIL-R2. As discussed above, Griffith and Lynch (Current Opinion in Immunology, 10:559-563, 1998) and Griffith et al. (J. Immunol. 162:2597, 1999) describe techniques for evaluating TRAIL receptor expression on cancer cells, and report their findings regarding expression of TRAIL-R1, R2, R3, and R4 for a number of different cancer cell lines.

### EXAMPLE 6

### Lysis of CMV-Infected Cells

Agonistic bispecific antibodies may be tested for cytotoxic effect on virally infected cells, by conventional assays such as the following.

Normal human gingival fibroblasts are grown to confluency on 24 well plates in 10% CO₂ and DMEM supplemented with 10% fetal bovine serum, 100 μg/ml streptomycin, and 100 μg/ml penicillin. To infect cells with cytomegalovirus (CMV), culture medium is aspirated and the cells are infected with CMV in DMEM with an approximate MOI (multiplicity of infection) of 5.

After two hours the virus-containing medium is replaced with DMEM, and the antibody of interest is added. After 24 hours the cells are stained with crystal violet dye as described (Flick and Gifford, 1984, supra). Stained cells are washed twice with water, disrupted in 200 μl of 2% sodium deoxycholate, diluted 5 fold in water, and the OD taken at 570 nm. Percent maximal staining was calculated by normalizing ODs to the sample that showed the greatest staining. Antibodies that kill CMV infected fibroblasts, without significant death of non-virally infected fibroblasts, are thus identified.

What is claimed is:

1. A bispecific antibody that binds TRAIL receptor 1 and TRAIL receptor 2.

2. A bispecific antibody of claim 1, wherein the antibody is a monoclonal antibody.

3. A bispecific antibody of claim 1, wherein the antibody induces death in a target cell selected from the group consisting of a cancer cell and a virally-infected cell.

4. A bispecific antibody of claim 2, wherein the antibody induces death in a target cell selected from the group consisting of a cancer cell and a virally-infected cell.

5. A bispecific antibody of claim 3, wherein the target cell is a virally-infected cell.

6. A bispecific antibody of claim 4, wherein the target cell is a virally-infected cell.

7. A bispecific antibody of claim 3, wherein the target cell is a cancer cell.

8. A bispecific antibody of claim 4, wherein the target cell is a cancer cell.

9. A bispecific antibody of claim 7, wherein the cancer cell is selected from the group consisting of leukemia, lymphoma, melanoma, breast carcinoma, colon carcinoma, and colorectal cancer cells.

10. A bispecific antibody of claim 8, wherein the cancer cell is selected from the group consisting of leukemia, lymphoma, melanoma, breast carcinoma, colon carcinoma, and colorectal cancer cells.

11. A method for killing cancer cells, comprising contacting cancer cells with a bispecific antibody of claim 7.

12. A method of claim 11, wherein the cancer cells are selected from the group consisting of leukemia, lymphoma, melanoma, breast carcinoma, colon carcinoma, and colorectal cancer cells.

13. A method for killing cancer cells, comprising contacting cancer cells with a bispecific antibody of claim 8.

14. A method of claim 13, wherein the cancer cells are selected from the group consisting of leukemia, lymphoma, melanoma, breast carcinoma, colon carcinoma, and colorectal cancer cells.

15. A method of claim 13, wherein the antibody comprises at least one Fc region.

16. A method of claim 13, wherein the antibody is a whole antibody.

17. A method for killing virally infected cells, comprising contacting virally infected cells with a bispecific antibody of claim 5.

18. A method for killing virally infected cells, comprising contacting virally infected cells with a bispecific antibody of claim 6.

19. A method of claim 17, wherein the cells are infected with human immunodeficiency virus (HIV).

20. A method of claim 18, wherein the cells are infected with human immunodeficiency virus (HIV).

21. A method of claim 18, wherein the antibody comprises at least one Fc region.

22. A method of claim 18, wherein the antibody is a whole antibody.

23. A method for killing cancer cells in vivo, comprising administering a bispecific antibody of claim 7 to a human who has cancer.

24. A method of claim 23, wherein the cancer cells are selected from the group consisting of leukemia, lymphoma, melanoma, breast carcinoma, colon carcinoma, and colorectal cancer cells.

25. A method for killing cancer cells in vivo, comprising administering a bispecific antibody of claim 8 to a human who has cancer.

26. A method of claim 25, wherein the cancer cells are selected from the group consisting of leukemia, lymphoma, melanoma, breast carcinoma, colon carcinoma, and colorectal cancer cells.

27. A method of claim 25, wherein the antibody comprises at least one Fc region.

28. A method of claim 25, wherein the antibody is a whole antibody.

29. A method of claim 23, wherein the antibody is co-administered with one or more additional anti-cancer agents.

30. A method for killing virally infected cells in vivo, comprising administering a bispecific antibody of claim 5 to a human who is infected with a virus.

31. A method for killing virally infected cells in vivo, comprising administering a bispecific antibody of claim 6 to a human who is infected with a virus.

32. A method of claim 30, wherein the virus is human immunodeficiency virus.

33. A method of claim 31, wherein the virus is human immunodeficiency virus.

34. A method of claim 31, wherein the antibody comprises at least one Fc region.

35. A method of claim 31, wherein the antibody is a whole antibody.

36. A method of claim 30, wherein the antibody is co-administered with one or more additional anti-viral agents.
